(19) United States
(12) Patent Application Publication
(10) Pub. No.: US 2006/0210548 A1
(43) Pub. Date: Sep. 21, 2006

(54) Method for reducing obstructive hydrocephalus

(76) Inventors: Jay M. Meythaler, Grosse Pointe Farms, MI (US); Winfield S. Fisher III, Birmingham, AL (US)

Correspondence Address: GIFFORD, KRASS, GROH, SPRINKLE, ANDERSON &;CITKOWSKI, P.C., P.O. BOX 7021, TROY, MI, 48007-7021, US

(21) Appl. No.: 10/516,899

(22) PCT Filed: Jun. 3, 2003
(86) PCT No.: PCT/US03/17271

§ 371 Date: Feb. 3, 2006

Related U.S. Application Data
(60) Provisional application No. 60/384,973, filed on Jun. 3, 2002.

(51) Int. Cl. A61K 38/48

(52) U.S. Cl. 424/94.63; 424/94.64

(57) ABSTRACT

A process for reducing cerebrospinal fluid flow obstruction includes the administration of a therapeutic dose of a clot-reducing agent to a subject having preconditions or obstructive hydrocephalus symptoms. The dose is maintained within the subject for a period of time sufficient to reduce cerebrospinal fluid flow obstruction. The clot-reducing agent includes a plasminogen activator, a defibrinogenic agent, an anticoagulant, a platelet inhibitor and a combination thereof. A commercial kit is provided, containing a clot-reducing agent, an administering apparatus, together with instructions for use of the kit.

### RELATED APPLICATION

This application claims priority of U.S. Provisional Patent Application Ser. No. 60/384,973 filed Jun. 3, 2002, which is incorporated herein by reference.

### FIELD OF THE INVENTION

The invention relates to use of pharmacologic agents in reducing cerebrospinal fluid (CSF) flow blockage. In particular, the invention relates to use of clot-reducing agents for reduction of obstructive hydrocephalus.

### BACKGROUND OF THE INVENTION

Obstructive hydrocephalus is defined as a pathology that results from obstruction of the flow of CSF. The consequence of such obstruction can be an increase in space occupied by ventricles or other CSF conduits which then impinge on brain tissues. One cause of hydrocephalus is hemorrhage. Following a hemorrhage, a blood clot may form and block a CSF conduit, thereby leading to obstructive hydrocephalus. If untreated, this blockage may quickly lead to death from excessive intracranial pressure.

Obstructive hydrocephalus is commonly treated by draining fluid from the cerebral ventricles or spinal canal. There are several problems with the current management of obstructive hydrocephalus. First, there is no evidence that temporary external ventricular drainage utilizing the current ventriculostomy catheters speeds clot resolution. Indeed, there is some evidence that external ventricular drainage may retard blockage resolution due to intraventricular hemorrhage or other blocking event. It is clear that external ventricular drainage must be maintained until the clot occluding the CSF conduits is resolved. Furthermore, external ventricular drainage alone is often inadequate therapy for obstructive hydrocephalus.

Thus, there is a continuing need for effective treatment of obstructive hydrocephalus and in particular, obstructions following intraventricular hemorrhage events.

### SUMMARY OF THE INVENTION

A process for reducing cerebrospinal fluid flow obstruction includes the administration of a therapeutic dose of a clot-reducing agent to a subject having preconditions or obstructive hydrocephalus symptoms. The dose is maintained within the subject for a period of time sufficient to reduce cerebrospinal fluid flow obstruction.

A commercial kit is provided, containing a clot-reducing agent, an administering apparatus, together with instructions for use of the kit. An administering apparatus is optionally a catheter for delivery of the clot-reducing agent to the CSF of a subject. In a further option, an administering apparatus is a syringe.

An inventive kit includes a clot-reducing agent chosen from the group including a plasminogen activator, a defibrinogenic agent, an anticoagulant, a platelet inhibitor and a combination thereof. A particularly preferred agent is an ancrod.

### DETAILED DESCRIPTION OF THE INVENTION

CSF is produced by the choroid plexus and flows through lateral ventricles, the foramen of Monro, the third ventricle, the aqueduct of Sylvius, the fourth ventricle, the subarachnoid spaces and to the arachnoid villi of the superior sagittal sinus. The arachnoid villi in the superior sagittal sinus are the primary site of CSF absorption into the venous bloodstream. If any of the above-mentioned spaces through which the CSF flows is occluded by a clot, for example after a hemorrhage into a ventricle, CSF reabsorption may be impeded and hydrocephalus results.

A blood clot can form when the coagulation cascade is activated in response to hemorrhage. The major components of a blood clot include fibrin and platelets. In one step of the coagulation cascade, fibrinogen is activated to form fibrin. Inactive fibrinogen is composed of polypeptide pairs designated α, β, and γ which are linked via disulfide bonds. Thrombin-mediated hydrolysis of fibrinogen produces fibrin monomers that can aggregate to form a fibrin clot. The clot is further cross linked by factor XIIIa, a transglutaminase, and aggregations of platelets and other factors form part of the clot mass. Platelet aggregation is typically mediated through a conformational change in a glycoprotein (GP) IIb/IIIa receptor.

Vascular obstruction can also be caused by blockage known as a thrombus. A thrombus is usually formed in absence of blood vessel rupture, for instance in response to plaque rupture. As used herein, the term “clot” refers both to the blood coagulation typically referred to as a clot and to a thrombus.

The term “clot-reducing agent” as used herein is intended to mean a protein or carbohydrate molecule, or combination thereof, such as a glycosylated protein, that decreases obstructive hydrocephalus. For example, a clot-reducing agent may decrease obstruction by reducing an existing clot via stimulation, direct or indirect, of clot lysis. A clot-reducing agent may further decrease obstruction by disfavoring clot formation.

Clots are degraded in the body by a process involving action of endogenous fibrinolytic clot-reducing agents. Typically, a serine protease, plasmin, which must be converted from its inactive form, plasminogen, is responsible for digesting fibrin and thereby reducing clots. Exogenous fibrinolytic clot-reducing agents, natural and synthetic, may be administered to an individual to stimulate clot degradation. For example, a plasminogen activator may be administered as a clot-reducing agent. Plasminogen activators illustratively include tissue plasminogen activator (tPA) and its recombinant variants known in the art such as alteplase, reteplase, saruplase, tenecteplase (TNK-ase) and lanoteplase described in Ross, Clinical Cardiology, 1999, 22:165. Plasminogen activators also include streptokinase, staphylokinase, urokinase, pro-urokinase and bat-PA. Plasminogen activators differ in the mechanism of their effect on plasminogen. For example, as described in Tsikouris and Tsikouris, alteplase, reteplase and tenecteplase directly cleave plasminogen to plasmin. However, streptokinase induces conformational changes in plasminogen that results in its having plasmin-like activity without cleavage. (Tsikouris, J P, Tsikouris, A P, A review of available fibrin-specific thrombolytic agents used in acute myocardial infarction. Pharmacotherapy 2001: 21(2):207-217.

Plasminogen is activated by enzyme cleavage to produce plasmin, which includes a heavy chain linked by disulfide bonds to a light chain. Plasminogen and plasmin may be cleaved to produce catalytically active enzymes having smaller size, for example see U.S. Pat. No. 4,774,087; U.S. Patent Application No. 20030096247; and Moroz, L A, Blood, 58:97-104 (1981). Such smaller enzymes and proenzymes are included in an inventive method as clot-reducing agents. Further, it is appreciated that enzyme-substrate complexes, such as a plasminogen/plasminogen activator complex, have activity as clot-reducing agents and as such these complexes are within the contemplated scope of the term “clot-reducing agent.”

A clot-reducing agent also includes a molecule that disfavors clot formation, such as an anticoagulant and a platelet inhibitor. A classic anticoagulant is a thrombin inhibitor. Thrombin usually cleaves fibrinogen to yield fibrin which may then be incorporated into a clot. Thrombin also activates various factors involved in clot formation, such as conversion of factor V to Va, factor VIII to VIIIa, factor XIII to XIIIa and activation of platelets. Thus, inhibition of thrombin, either directly or indirectly, inhibits clot formation. Examples of thrombin inhibitors include the coumarin derivatives bishydroxycoumarin (Dicumarol) and warfarin (Coumadin); thrombate and lepirudin. Further inhibitors include hirudin, bivalirudin, melagatran and H376/95. Argatroban, a synthetic arginine derivative that acts as a direct thrombin inhibitor, also known as Novastan®, Texas Biotechnology Corp., is a particularly preferred clot-reducing agent in a method of the present invention. Further information on argatroban and related agents, such as efegatran, inogatran and napsagatran, may be found in Swan, S K and Hursting, M J, The Pharmacokinetics and Pharmacodynamics of Argatroban: Effects of Age, Gender, and Hepatic or Renal Dysfunction, Pharmacotherapy, 20(3):318-329, 2000; Hauptmann J, Pharmacokinetics of an Emerging New Class of Anticoagulant/Antithrombotic Drugs. A Review of Small-Molecule Thrombin Inhibitors, Eur J Clin Pharmacol, 57(11):751-8, 2002.

Further examples of clot-reducing agents are found in Colman R W, Hirsh J, Marder V J, Salzman E W, eds. Hemostasis and Thrombosis: Basic Principles and Clinical Practice. 3rd ed. Philadelphia, Pa.: JB Lippincott Co.; 1994:1638-1660; Hirsh, J. New Anticoagulants, Am Heart J 2001: 142(2):s3-s8 and Tsikouris, J P, Tsikouris, A P, A review of available fibrin-specific thrombolytic agents used in acute myocardial infarction. Pharmacotherapy 2001: 21(2):207-217.

A number of toxins produced by reptiles have been found to have effects on coagulation in humans and other mammals. A subset of these decrease coagulation and are therefore clot-reducing agents.

Clot reduction is further achieved by administration of an agent that inhibits clot formation by inhibiting production of clot forming components. For instance, fibrinogen may be effectively decreased in the plasma, making it unavailable as a source for fibrin. Such a clot-reducing agent is referred to as defibrinogenic. Examples of such defibrinogenic clot-reducing agents are included in a subset of reptile venoms that include clot-reducing agents that are defibrogenic agents. These include calobin I, calobin II, batroxobin, gyroxin, acutin, Venzyme, asperase, reptilase, botropase, defibrase, crotalase, flavoxobin and gabonase. Further examples of reptile toxins that are clot-reducing agents are found in the references: Pirkle, H and I Theodor: Thrombin-like enzymes, in “Snake Venom Enzymes,” G S Bailey (ed.), Alaken, Inc., Fort Collins, Colo., 1998; Snake Venom Fibrinogenolytic and Fibrinolytic Enzymes: An Updated Inventory, F S Markland, Jr., Thrombosis, March 1998; Thrombin-like Enzymes from Snake Venoms: An Updated Inventory, H Pirkle, Thrombosis, March 1998, as well as in H Pirkle, K Stocker, Thrombin-like enzymes from snake venoms: an inventory. For the Subcommittee on Nomenclature of Exogenous Hemostatic Factors of the Scientific and Standardization Committee of the International Society on Thrombosis and Haemostasis. Thrombosis and Haemostasis, 65(4):444-450, 1991 and H Pirkle, Thrombin-like venom enzymes: structure and function. Adv Exp Med Biol 281:165-175, 1990.

Clot-reducing agents are produced in numerous species of snake including Agkistrodon acutus, Agkistrodon contortrix contortrix, Agkistrodon halys pallas, Bothrops asper, Bothrops insularis, Bothrops jararaca, Lachesis muta muta, Crotalus adamanteus, Bothrops atrox, Bothrops moojeni, Bothrops marajoensis, Bothrops maranhao, Bothrops asper, Bothrops pradoi, Crotalus atrox, Crotalus durissus terrificus, Trimeresurus flavoviridis, Trimeresurus gramineus, Viper aspis, Viper berus, Denisonia superba, Notechis scutatus, Bitis gabonica and Pseudechis porphyriacus. In addition, synthetic versions of venom clot-reducing agents such as recombinant versions and mutant variants thereof are preferred in an inventive method. Venoms from other reptiles are useful as clot-reducing agents where an effect on coagulation is shown using standard coagulation assays known to those skilled in the art. An example of such an assay is detailed in U.S. Pat. No. 4,154,656.

A particularly preferred defibrinogenic snake venom clot-reducing agent is ancrod. Ancrod is a protein isolated from the venom of the Malayan pit viper, Calloselasma rhodostoma or Agkistrodon rhodostoma. This protein is a glycosylated serine protease that cleaves fibrinogen as described in Wright J G and Geroulakos G, Seminars in Vascular Surg 1996; 9: 315-328 and in Soutar R L, Ginsberg J S, Crit Rev Oncol Hematol, 1993, 15: 23-33. Ancrod may have further actions on molecules of the fibrinolytic pathway that contribute to its clot-reducing properties as detailed in Wright and Geroulakos (supra). Ancrod is available commercially from Knoll GmbH, Germany. While not wishing to be bound by theoretical considerations, it has been hypothesized that ancrod acts to decrease fibrinogen in the circulation by cleavage to yield fibrin monomers that do not form usual fibrin clots due to an inability to bind other fibrin monomers. In this context, ancrod is believed to preferentially cleave A-fibrinopeptides rather than B-fibrinopeptides from fibrinogen.

Defibrinogenic agents from reptile venoms are distinct in their enzymatic effects on fibrinogen. For example, atroxase, a protease isolated from Crotalus atrox cleaves A and B chains from fibrinogen but not the G chain. (Willis and Tu, Biochemistry, 1988, 27:4769-77.) In contrast, the venom of Naja nigricollis contains an enzyme that preferentially cleaves the A chain of fibrinogen. (Evans, H J, Biochem Biophys Acta, 1984, 802:49-54.) Thus, different classes of defibrinogenic agents from venoms are defined by their proteolytic action. Further examples of this classification of venom enzymes is found in H Pirkle, K Stocker, Thrombin-like enzymes from snake venoms: an inventory. For the Subcommittee on Nomenclature of Exogenous Hemostatic Factors of the Scientific and Standardization Committee of the International Society on Thrombosis and Haemostasis. Thrombosis and Haemostasis, 65(4):444-450, 1991 and H Pirkle, Thrombin-like venom enzymes: structure and function. Adv Exp Med Biol 281:165-175, 1990.

In another embodiment of a process of the present invention, batroxobin, especially from Bothrops atrox, Bothrops moojeni, Bothrops maranhao is a clot-reducing agent.

A clot-reducing agent from venom is particularly preferred since only small volumes are required to be administered. Typically, less than one milliliter is administered into the cerebral spinal fluid.

In addition to the action of a clot-reducing agent in breaking down a clot, a subset of clot-reducing agents acts to reduce clots by preventing clotting or preventing further clotting. In this context, administration of an anticoagulant is optionally included in a method to reduce obstructive hydrocephalus. An anticoagulant includes low molecular weight heparins such as ardeparin, dalteparin, and enoxaparin as well as heterogeneous heparin having higher molecular weight components, as is known in the art and as described in Lane D A, Lindahl U, eds. Heparin: Chemical and Biological Properties, Clinical Applications. London, England: Edward Arnold; 1989. Commercial names for low molecular weight heparins include Fragmin (dalteparin sodium—MW about 2000-9000 daltons), Lovenox (enoxaparin sodium—MW about 2000-8000 daltons), Normiflo (ardeparin sodium—MW about 5650-6350 daltons). Orgaran (danaparoid sodium) is representative of a non-heparin antithrombotic clot-reducing agent, being a composition of low molecular weight sulfated glycosaminoglycuronans. The group of clot-reducing agents also includes platelet inhibitors. Platelet inhibitors may oppose platelet aggregation at any of a number of steps in the clot-forming process including inhibiting of platelet activation and inhibiting platelet recruitment. Platelet inhibitors block a change in conformation of the GPIIb/IIIa receptor that usually occurs due to the action of thrombin. Antagonists include thromboxane A2 synthesis blockers such as aspirin, inhibitors of ADP binding such as ticlopidine and clopidogrel, and inhibitors of binding to GPIIb/IIIa receptor such as tirofiban and eptifibatide. The mechanism of action of some platelet aggregation inhibitors, such as dipyridamole, is incompletely characterized, but this does not limit use in a method of the present invention.

It will be recognized by one of skill in the art that neither the mechanism by which a clot-reducing agent acts nor the step in the coagulation cascade that it affects is limiting in a process of the present invention as long as the clot-reducing agent acts to decrease obstructive hydrocephalus:

It is appreciated that administration of thrombin inhibitors inhibits inflammation and that administration of thrombin inhibitors as described herein for clot-reduction and reduction of obstructive hydrocephalus will also have beneficial anti-inflammatory effects (U.S. Pat. No. 6,232,315).

### Methods of Treatment

A clot-reducing agent may be introduced into the CSF by routes illustratively including intraventricular and intrathecal. The agent is also administered systemically, for example intravenously, where the agent passes into the CSF. Optionally, an agent or combination of agents is administered both into the CSF and into a blood vessel to achieve a synergistic effect in the treatment of obstructive hydrocephalus.

Hemorrhage of a blood vessel is a predisposing factor to obstructive hydrocephalus. In particular, bleeding into a CSF conduit creates a risk of obstructive hydrocephalus as a result of subsequent clotting and blockage of a CSF conduit. An individual is typically treated following a hemorrhage that results in clotting which obstructs CSF flow. Further, an individual may be treated according to an inventive method in order to prevent clotting which might cause obstruction of CSF flow where indicated. For example, in the case in which an individual has had a cerebral hemorrhage, delivery of a clot-reducing agent into the CSF may be desirable to prevent CSF flow obstruction.

The exact amount of the clot-reducing agent required as a therapeutic dose and the therapeutic amount maintained in the subject to reduce CSF flow obstruction will vary from subject to subject, depending on the age, weight and general condition of the subject, the severity of the condition that is being treated, the location and size of the clot, the particular clot-reducing agent or combination of agents used, the mode of administration, and the like. An appropriate therapeutic dose and therapeutic amount to be maintained in the subject may be determined by one of ordinary skill in the art using only routine experimentation given the teachings herein. For example, a clot-reducing agent is administered and the level of the agent in the CSF is monitored by periodic withdrawal of CSF and assay for the agent. This is accomplished by a method illustratively including withdrawal of CSF through an intraventricular or intrathecal catheter, or by lumbar puncture. In order to determine an appropriate period of time sufficient to reduce CSF flow obstruction/obstructive hydrocephalus, a subject is monitored by techniques known to those skilled in the art. For example, images of an obstructed area are obtained by MRI and monitored over time of treatment. Further, levels of an indicator of clot lysis are assayed to determine the extent of action of the clot-reducing agent. Other techniques for determining the extent of obstruction and its reduction are known in the art, for example as detailed in Sanders, R C, Clinical Sonography: A Practical Guide 3^{rd} Edition, Lippincott and Squire, L F, Novelline, R A; Squire's Fundamentals of Radiology, 1997, Fifth Edition. In general, a therapeutic dose is ½ to 1/1000 of the dose of a clot-reducing agent given systemically. Preferably, a therapeutic dose is ¼ to 1/500 of the systemic dose. More preferably still, a therapeutic dose is ⅙ to 1/250 of the systemic dose.

Exemplary doses and modes of administration include: Ancrod: 2-5 IU (international units) bolus dose q (every) 12 hours for 48 hours via micropore filter through a catheter, the catheter is to remain closed for one hour not allowing CSF drainage unless the ICP reaches >20 mm Hg; Urokinase: 5000-25000 IU bolus dose q (every) 12 hours for 48 hours, via micropore filter through via a catheter, the catheter is to remain closed for one hour not allowing CSF drainage unless the ICP reaches >20 mm Hg. See for example Naff et al.; Streptokinase: 100,000-250,000 IU in 1-2 cc. Preservative free saline bolus dose q (every) 12 hours for 48 hours, via micropore filter through via a catheter, the catheter is to remain closed for one hour not allowing CSF drainage unless the ICP reaches >20 mm Hg; Argatroban 175-250 μg (microgram) bolus dose q (every) 12 hours for 48 hours, via micropore filter through via a catheter, the catheter is to remain closed for one hour not allowing CSF drainage unless the ICP reaches >20 mm Hg.

A combination of clot-reducing agents may be used in a method of the present invention and any combination of clot-reducing agents that are compatible with each other is administrable to reduce obstructive hydrocephalus. In some embodiments, it is preferred to co-administer clot-reducing agents having complementary actions on a clot. For example, it may be preferable to co-administer a defibrinogenic agent and a plasminogen activator. Co-administration indicates administration to an individual patient and delivery of the clot-reducing agents selected may be simultaneous or sequential.

Depending on the intended mode of administration, the clot-reducing agent can be in a pharmaceutical composition in the form of solid, semi-solid or liquid dosage forms, such as, for example, liquids, or suspensions. The dose may be given in unit dosage form suitable for single administration of a precise dosage.

Preferably a clot-reducing agent is given by an intraventricular or intrathecal catheter. Administration via this route allows delivery of small volumes of a therapeutic agent to the vicinity of the desired action and lessens the exposure of other tissues to the drug. A preferred CNS catheter assembly includes branches and a main body which defines at least one lumen therethrough. The branches and main catheter body are preferably tubular in shape. The branch includes a proximally disposed opening or port which provides access to the lumen. The branch is preferably designed for the introduction or delivery of drugs therethrough. The branch can also include a connector or adapter disposed directly adjacent to or about the proximal opening or port which allows for the connection or attachment of a fluid delivery device, such as a syringe, to the branch for delivery of a therapeutic agent and/or a drug therethrough. Particularly preferred is administration by the catheter described in International Application PCT/US00/05740 which is incorporated herein by reference. A catheter is inserted into either the spinal canal or a ventricle of the brain in order to remove cerebrospinal fluid (CSF), monitor intracranial pressure (ICP), and/or deliver therapeutic agents and/or drugs intrathecally and/or intraventricularly, directly into the cerebrospinal fluid.

A therapeutic amount is maintained in the subject by, for example, continuous administration by an intraventricular or intrathecal catheter. The clot-reducing agent is administered in combination with a pharmaceutically acceptable carrier and, in addition, may include other medicinal agents, pharmaceutical agents, carriers, or dilutants. By “pharmaceutically acceptable” is meant a material that is not biologically or otherwise undesirable, which can be administered to an individual along with the selected clot-reducing agent without causing significant undesirable biological effects or interacting in a deleterious manner with any of the other components of the pharmaceutical composition in which it is contained.

Compositions suitable for administration of a clot-reducing agent to CSF may comprise physiologically acceptable sterile aqueous or non-aqueous solutions, dispersions, suspensions or emulsions, and sterile powders for reconstitution into sterile administrable solutions or dispersions. Examples of suitable aqueous and non-aqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like.

Liquid dosage forms include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

The term “pharmaceutically acceptable salts, esters, amides, and prodrugs” as used herein refers to those carboxylate salts, amino acid addition salts, esters, amides, and prodrugs of the clot-reducing agents which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate and laurylsulphonate salts, and the like. These may include cations based on the alkali and alkaline earth metals, such as sodium, lithium, potassium, calcium, magnesium, and the like, as well as non-toxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, S M Berge, et al., “Pharmaceutical Salts,” J Pharm Sci, 1977; 66:1-19 which is incorporated herein by reference.)

The term “prodrug” refers to compounds that are rapidly transformed in vivo to yield the parent compound of the above formula, for example, by hydrolysis in blood. A thorough discussion is provided in T Higuchi and V Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

A clot-reducing agent is administered to a patient at appropriate dosage levels for reducing CSF flow obstruction due to obstructive hydrocephalus. The dosage depends on a number of factors including the requirements of the patient, the severity of the condition being treated, and the pharmacological activity of the compound being used. In general, the dosage to reduce CSF flow obstruction will be under one-eighth of the dose introduced into the venous or arterial system for reduction of acute clotting in a blood vessel. The determination of optimum dosages for a particular patient is well known to those skilled in the art.

The present invention provides a kit containing a clot-reducing agent and including any reagents or components necessary for the administration of the compounds, together with instructions for use of the kit. An inventive kit includes an administering apparatus for delivery of a clot-reducing agent. The kit optionally includes a catheter for delivery of the clot-reducing agent to the CSF of a subject. A preferred catheter for inclusion in a kit is described herein and in International Application PCT/US00/05740. In a further option, an administering apparatus is a syringe.

### REFERENCES

Rolli, M L Advances in the treatment of adult intraventricular hemorrhage. Seminars in Neurosurgery. (2000) 11:27-39.
Kassell N F, Torner J C, Adams I P Jr. Antifibrinolytic therapy in the acute period following aneurysmal subarachnoid hemorrhage. Preliminary observations from the Cooperative Aneurysm Study. J. Neurosurg. 1984 August; 61(2):225-30.
Findlay J, Weir B, Steinke D, et al. Effect of intrathecal thrombolytic therapy on subarachnoid clot and chronic vasospasm in a primate model of SAH. J. Neurosurg. 1988 November; 69(5):723-35.
Findlay, J, Weir, B, Gordon, P, et al., Safety and efficacy of intrathecal thrombolytic therapy in a primate model of cerebral vasospasm. (1989) Neurosurgery 24:491-498.
Findlay, J M, Weir, B K, Kanamaru, K, et al., Intrathecal fibrinolytic therapy after subarachnoid hemorrhage: Dosage study in a primate model and review of the literature. (1989) Can. J. Neurol. Sci. 16:28-40.
Findlay, J M, Weir, B K, Kassell, N F, Disney, L B and Grace, M G. Intracisternal recombinant tissue plasminogen activator after aneurysmal subarachnoid hemorrhage. (1991) J. Neurosurgery 75:181-188.
Zabramski, J M, Spetzler, R F, Lee K S, et al. Phase I trial of tissue plasminogen activator for the prevention of vasospasm in patients with aneurysmal subarachnoid hemorrhage. (1991) J. Neurosurgery 75:189-196.
Kodama, N, Sasaki, T and Kawakami, M. Prevention of vasospasm: Cisternal irrigation therapy with urokinase ad ascorbic acid. In: Sano, K, Takakura, K, Kassell, N, eds. Cerebral Vasospasm: Proceedings of the IVth International Conference on Cerebral Vasospasm, Tokyo, 1990, Tokyo, Japan, University of Tokyo Press, pp. 292-296.
Mizoi, K, Yoshimoto, T, Takahashi, A, Fujiwara, S, Koshu, K and Sugawara, T. Prospective study on the prevention of cerebral vasospasm by intrathecal fibrinolytic therapy with tissue-type plasminogen activator. (1993) J. Neurosurg. 78:430-437.
Mizoi, K, Yoshimoto, T, Fujiwara, S, and Sugawara, T., Takahashi, A, and Koshu, K. Prevention of vasospasm by clot removal and intrathecal bolus injection of tissue-type plasminogen activator: Preliminary report. (1991) Neurosurgery 28:807-812.
Sasaki, T, Ohta, T, Kikuch, H, et al., Preliminary clinical trial of intrathecal rt-PA (TD-2061) for the prevention of cerebral vasospasm in patients with aneurysmal subarachnoid hemorrhage. (1992) No To Shinkei 44:1001-1008.
Usui, M, Saito, N, Hoya, K and Todo, T. Vasospasm prevention with postoperative intrathecal thrombolytic therapy: A retrospective comparison of urokinase tissue plasminogen activator, and cisternal drainage alone. (1994) Neurosurgery 34:235-244.
Findlay, J M, Kassell, N F, Weir, B K, et al., A randomized trial of inoperative intracisternal tissue plasminogen activator for the prevention of vasospasm. (1995) Neurosurgery 37:168-176.
Findlay, J M, Grace, M G and Weir, B K. Treatment of intraventricular hemorrhage with tissue plasminogen activator. (1993) Neurosurgery 32:941-947.
Mayfrank, L, Lippitz, B, Groth, M, Bertalanffy, H and Gilsbach, J M. Effect of recombinant tissue plasminogen activator on clot lysis and ventricular dilatation in the treatment of severe intraventricular hemorrhage. (1993) Acta Neurochir. 122:32-38.
Rohde, V, Schaller, C and Hassler, W E. Intraventricular recombinant tissue plasminogen activator for lysis of intraventricular hemorrhage. (1995) J. Neurol. Neurosurg. Psychiatry. 58:447-451.
Shen, P H, Matsuoka, Y, Kawajiri, K, et al. Treatment of intraventricular hemorrhage using urokinase. (1990) Neurol. Med. Chir. (Tokyo) 30:329-333.
Todo, T, Usui, M and Takakura, K. Treatment of severe intraventricular hemorrhage by intraventricular infusion of urokinase. (1991) J. Neurosurg. 74:81-86.
Schwarz, S, Schwab, S, Steiner, H H and Hacke, W. Secondary hemorrhage after intraventricular fibrinolysis: A cautionary note: A report of two cases. (1998) 42:659-662.
Akdemir H, Selcuklu, A, Pasoglu, A, Oktem, I S and Kavuncu, I. Treatment of severe intraventricular hemorrhage by intraventricular infusion of urokinase. (1995) Neurosurg. Rev. 18:95-100.
Coplin, W M, Vinas, F C, Agris, J M, et al. A cohort study of the safety and feasibility of intraventricular urolcinase for nonaneurysmal spontaneous intraventricular hemorrhage. (1998) Stroke 29:1573-1579.
Grabb, P A. Traumatic intraventricular hemorrhage treated with recombinant tissue plasminogen activator. Technical case report. (1998) Neurosurgery 43:966-969.
Goh, K Y and Poon, W S. Recombinant tissue plasminogen activator for the treatment of spontaneous adult intraventricular hemorrhage. (1998) Surg. Neurol. 50:526-531.
Naff, N J, Tuhrim, S, Williams, M S and Hanley, D F. Intraventricular urokinase injections speed blood clot resolution in IVH: A quantitative analysis. Abstract. Joint Section on Cerebrovascular Surgery Annual Meeting. Nashville Tenn.: 1999.
Naff, N J, Carhuapoma J R, Williams M A, Bhardwaj, A, Ulatowski J A, Bederson J, Bullock R, Schmutzhard E, Pfasuler B, Keyl P M, Tuhrim S,
Hanley D F., Treatment of intraventricular hemorrhage with urokinase. Stroke 2000; 31:841-847.
Sherman, D G, Atkinson, R P, Chippendale, T, et al. Intravenous ancrod for treatment of acute ischemic stroke. The STAT Study: A randomized control trial. (2000) JAMA 283: 2395-2403.
Wilcox, R G, von der Lippe, G, Olsson, C G, et al. Trial of tissue plasminogen activator for mortality reduction in acute myocardial infarction: Anglo-Scandinavian study of early thrombolysis (ASSET) Lancet 1988; 2:525-30.
AIMS Trial Study Group. Long-term effects of intravenous anistreplase in acute myocardial infarction: final report of the AIMS study. Lancet 1990; 335:427-31.
Gruppo Italiano per lo Studio della Streptochinasi nell'Infarto Miocardico (GISSI). Effectiveness of intravenous thrombolytic treatment in acute myocardial infarction. Lancet 1986; 1:397-402.
ISIS-2 (Second International Study of Infarct Survival) Collaborative Group. Randomized trial of intravenous streptokinase, oral aspirin, both or neither among 17,187 cases of suspected acute myocardial infarction: ISIS-2. Lancet 1988; 2:349-60.
A. Gomes and Pallabi De, Hannahpep: A novel fibrinolytic peptide from the Indian king cobra (Ophiophagus hannah) venom. (1999) Biochem. Biophys. Res. Comm. 266:488-491.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present methods, procedures, treatments, molecules, apparatus and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A process of reducing cerebrospinal fluid flow obstruction comprising:
administering a therapeutic dose of a clot-reducing agent to a subject having preconditions or obstructive hydrocephalus symptoms; and
maintaining a therapeutic amount of the clot-reducing agent within the subject for a period of time sufficient to reduce cerebrospinal fluid flow obstruction.

2. The process of claim 1 wherein the administering is by catheter.

3. The process of claim 1 wherein the administering is by a device selected from the group consisting of: intrathecal catheter, intraventricular catheter and an injection.

4. The process of claim 1 wherein the clot-reducing agent is selected from the group consisting of: a plasminogen activator, a defibrinogenic agent, an anticoagulant, a platelet inhibitor and a combination thereof.

5. The process of claim 4 wherein the plasminogen activator is selected from the group consisting of: alteplase, reteplase, saruplase, tenecteplase, lanoteplase, bat-PA, a combination thereof, a functional fragment thereof, a pharmacologically acceptable salt, ester, amide, or prodrug thereof.

6. The process of claim 4 wherein the plasminogen activator is tissue plasminogen activator, a functional fragment thereof, a pharmacologically acceptable salt, ester, amide, or prodrug thereof.

7. The process of claim 4 wherein the plasminogen activator is selected from the group consisting of: streptokinase, staphylokilnase, a combination thereof, a functional fragment of either streptokinase or staphylokinase, a pharmacologically acceptable salt of either streptokinase or staphylokinase, ester of either streptokinase or staphylokinase, amide of either streptokinase or staphylokinase, or prodrug of either streptokinase or staphylokinase.

8. The process of claim 4 wherein the plasminogen activator is selected from the group consisting of: urokinase and pro-urokinase, a combination thereof, a functional fragment of either urokinase or pro-urokinase, a pharmacologically acceptable salt of either urokinase or pro-urokinase, ester of either urokinase or pro-urokinase, amide of either urokinase or pro-urokinase, or prodrug of either urokinase or pro-urokinase.

9. The process of claim 4 wherein the defibrinogenic agent is a natural or synthetic reptile peptide, a combination thereof, a functional fragment thereof, a pharmacologically acceptable salt, ester, amide, or prodrug thereof.

10. The process of claim 9 wherein the reptile peptide is a snake venom enzyme, a functional fragment thereof, a pharmacologically acceptable salt, ester, amide, or prodrug thereof.

11. The process of claim 9 wherein the snake venom enzyme is selected from the group consisting of calobin I, calobin II, gyroxin, acutin, venzyme, asperase, reptilase, botropase, defibrase, crotalase, flavoxobin, gabonase, hannahpep, a combination thereof, a functional fragment thereof, a pharmacologically acceptable salt, ester, amide, or prodrug thereof.

12. The process of claim 4 wherein the defibrinogenic agent is ancrod, a functional fragment thereof, a pharmacologically acceptable salt, ester, amide, or prodrug thereof.

13. The process of claim 4 wherein the defibrinogenic agent is batroxobin, a functional fragment thereof, a pharmacologically acceptable salt, ester, amide, or prodrug thereof.

14. The process of claim 4 wherein the defibrinogenic agent is argatroban, a functional fragment thereof, a pharmacologically acceptable salt, ester, amide, or prodrug thereof.

15. The process of claim 4 wherein the anticoagulant is selected from the group consisting of: heparin, a thrombin inhibitor and a combination thereof.

16. The process of claim 15 wherein the thrombin inhibitor is selected from the group consisting of: a coumarin derivative, thrombate, lepirudin, hirudin, bivalirudan, melagatran and H376/95.

17. The process of claim 4 wherein the anticoagulant is a low molecular weight heparin.

18. The process of claim 4 wherein the platelet inhibitor is a GPIIb/IIIa antagonist.

19. The process of claim 4 wherein the platelet inhibitor inhibits thromboxane A2 synthesis.

20. The process of claim 4 wherein the platelet inhibitor is aspirin, a pharmacologically acceptable salt, ester, amide, or prodrug thereof.

21. The process of claim 4 wherein the platelet inhibitor is selected from the group consisting of: ticlopidine and clopidogrel.

22. The process of claim 4 wherein the platelet inhibitor is selected from the group consisting of: tirofiban and eptifibatide.

23. The process of claim 4 wherein the platelet inhibitor is dipyridamole.

24. A process of reducing cerebrospinal fluid flow obstruction comprising:
administering a therapeutic dose of a clot-reducing agent comprising ancrod to a subject having obstructive hydrocephalus; and
maintaining a therapeutic amount of the clot-reducing agent comprising ancrod within the subject for a period of time sufficient to reduce cerebrospinal fluid flow obstruction.

25. A process of reducing cerebrospinal fluid flow obstruction comprising:
administering a therapeutic dose of a clot-reducing agent comprising batroxobin to a subject having preconditions or symptoms of obstructive hydrocephalus; and
maintaining a therapeutic amount of the clot-reducing agent comprising batroxobin within the subject for a period of time sufficient to reduce cerebrospinal fluid flow obstruction.

26. A commercial kit for reducing obstructive hydrocephalus comprising:
a clot-reducing agent; and
instructions for use in reducing obstructive hydrocephalus.

27. The commercial kit of claim 26 further comprising a catheter for delivery of the clot-reducing agent to the cerebrospinal fluid of a subject.

28. The commercial kit of claim 26 wherein the clot-reducing agent is selected from the group consisting of: a plasminogen activator, a defibrinogenic agent, an anticoagulant, a platelet inhibitor and a combination thereof.

29. The commercial kit of claim 26 wherein the plasminogen activator is selected from the group consisting of: tissue plasminogen activator, alteplase, reteplase, saruplase, tenecteplase, lanoteplase, streptokinase, staphylokinase, urokinase, pro-urokinase and bat-PA.

30. The process of claim 26 wherein the anticoagulant is selected from the group consisting of: heparin, a thrombin inhibitor and a platelet inhibitor.

31. The commercial kit of claim 26 wherein the clot-reducing agent is ancrod.

32. The commercial kit of claim 26 wherein the clot-reducing agent is batroxobin.

33. The commercial kit of claim 26 wherein the clot-reducing agent is argatroban.

34. The commercial kit of claim 26 wherein the clot-reducing agent is streptokinase.

35. The commercial kit of claim 26 wherein the clot-reducing agent is urokinase.

36. A process of reducing cerebrospinal fluid flow obstruction substantially as described herein.

37. A commercial kit for reducing obstructive hydrocephalus substantially as described herein.

38. A process of clot-reducing agent delivery substantially as described herein.
